(19) United States
(12) Patent Application Publication
(10) Pub. No.: US 2008/0300628 A1
(43) Pub. Date: Dec. 4, 2008

(54) Medical Devices

(75) Inventor: Brian A. Ellingwood, Sunnyvale, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 11/757,108

(22) Filed: Jun. 1, 2007

(51) Int. Cl. A61B 17/08

(52) U.S. Cl. 606/221

(57) ABSTRACT

A medical system may include a medical device, such as a medical connector. The medical connector may be used to close an opening formed in (and/or interconnect portions of) one or more bodily structures. The medical connector may be sized and configured to resiliently move to a fastening position. The medical connector may be sized and configured to penetrate a portion of a wall of a lumen, such as a blood vessel. For example, medical connector may be sized and configured to initially penetrate a portion of the wall disposed between the wall's inner and outer surfaces.

### BACKGROUND OF THE INVENTION

1. Field of the Invention

The present invention generally relates to medical devices.

2. Background Technology

Intravascular medical procedures may be diagnostic or therapeutic in nature and commonly involve, for example, the insertion, use, and removal of any of a variety of devices to diagnose or treat a medical condition. For example, during such procedures, an opening may be made in a patient's blood vessel, and an introducer sheath may be used to insert a device (such as a catheter or stent) into the vessel. The introducer sheath is typically configured to help reduce blood loss during the procedure. Later, the device and the introducer sheath may be removed, leaving the opening in the vessel wall. If the opening is left unsealed, blood may escape and enter into the surrounding body cavities and tissue. Where excessive blood escapes, the effectiveness of the medical procedure may be compromised and complications may arise.

One method used to avoid excessive bleeding is to apply pressure to the affected area, for example, using manually applied pressure or using pressuring-applying devices, such as a sandbag, a bandage, or a clamp. This method attempts to minimize such bleeding until the natural clotting process stops the bleeding. (The stoppage of bleeding is commonly referred to as “hemostasis.”) Unfortunately, the clotting process may take a significant amount of time (up to two hours or more, in some cases), and a medical attendant may have to repeatedly monitor the patient throughout that time period. This may increase the cost of the medical procedure. Moreover, the pressure-application process's effectiveness can be compromised unless the patient remains motionless, which may require immobilization of the patient. Unfortunately, immobilization may be uncomfortable for the patient, especially given the significant amount of time that may be necessary to achieve hemostasis. In addition, given this significant amount of time, a patient may have to remain longer at a medical facility, thus further increasing the cost of the medical procedure.

Another method used to avoid excessive bleeding is to seal a vascular opening with a plug, such as a collagen plug. For example, a plug may be deployed into the vascular opening through an introducer sheath. When the plug is deployed, the blood or other body fluids may cause the plug to swell, which may block the opening and facilitate hemostasis. Such plugs may, however, be difficult to properly position in the vessel. An improperly positioned plug may undesirably block the flow of fluid in the vessel. Moreover, an improperly positioned plug may release into the blood stream where it might float downstream and potentially lodge in and/or obstruct blood flow.

### SUMMARY

A need therefore exists for medical devices that eliminate or reduce the disadvantages and problems listed above and/or other disadvantages and problems.

One aspect is a medical connector that may be used to close an opening formed in (and/or interconnect portions of) one or more bodily structures. The medical connector may include one or more penetrating members, which may be sized and configured to penetrate various portions of the one or more bodily structures.

Another aspect is a medical system that may include a medical connector. The medical connector may include at least one elongated member. The medical connector may be sized and configured to resiliently move from an everted position to a fastening position. The at least one elongated member may be arranged in a generally ring-shaped configuration when the medical connector is in the fastening position. The at least one elongated member may include a plurality of elongated members, which may be arranged in a generally sphere-shaped configuration when the medical connector is in the fastening position. The medical system may include a guide that may be sized and configured to retain the medical connector in the everted position. The medical system may include a biasing member that may be sized and configured to move the medical connector to a location at which the guide no longer retains the medical connector in the everted position and the medical connector resiliently moves to the fastening position. The medical system may include an extension that may be movable between a retracted position and an extended position to locate an opening in a patient's lumen. The medical system may include an extension that may be movable between a retracted position and an extended position, and the extension may be sized and configured to engage an inner surface of a wall of a patient's lumen when in the extended position. The at least one elongated member may include a first penetrating member and a second penetrating member, which may face generally towards each other when the medical connector is in the fastening position and may face generally away from each other when the medical connector is in the everted position. The at least one elongated member may include a first end and a second end, which may face generally towards each other when the medical connector is in the fastening position and may face generally away from each other when the medical connector is in the everted position. The at least one elongated member may include a first penetrating member and a second penetrating member, which may overlap when the medical connector is in the fastening position. The at least one elongated member may include a first penetrating member and a second penetrating member, and the first penetrating member may contact the second penetrating member when the medical connector is in the fastening position. The at least one elongated member may include a first end and a second end, which may overlap when the medical connector is in the fastening position. The at least one elongated member may include a first end and a second end, and the first end may contact the second end when the medical connector is in the fastening position.

Yet another aspect is a medical system that may include a medical connector. The medical connector may include at least one elongated member. The at least one elongated member may include a first penetrating member and a second penetrating member. The medical connector may be sized and configured to resiliently move from a first position to a second position. The first and second penetrating members may face generally towards each other when the medical connector is in the second position. The first and second penetrating members may face generally away from each other when the medical connector is in the first position. The medical system may include a guide that may be sized and configured to retain the medical connector in the first position. The medical system may include a biasing member that may be sized and configured to move the medical connector to a location at which the guide no longer retains the medical connector in the first position and the medical connector resiliently moves to the second position. The medical system may include an extension that may be movable between a retracted position and an extended position to locate an opening in a patient's lumen. The medical system may include an extension that may be movable between a retracted position and an extended position, and the extension may be sized and configured to engage an inner surface of a wall of a patient's lumen when in the extended position. The at least one elongated member may be arranged in a generally ring-shaped configuration when the medical connector is in the second position. The at least one elongated member may include a plurality of elongated members; and the plurality of elongated members may be arranged in a generally sphere-shaped configuration when the medical connector is in the second position. The first and second penetrating members may overlap when the medical connector is in the second position. The first penetrating member may contact the second penetrating member when the medical connector is in the second position.

Still another aspect is a medical system that may include a medical connector. The medical connector may include at least one elongated member. The at least one elongated member may include a first end and a second end. The medical connector may be sized and configured to resiliently move from a first position to a second position. The first and second ends may face generally towards each other when the medical connector is in the second position. The first and second ends may face generally away from each other when the medical connector is in the first position. The medical system may include a guide that may be sized and configured to retain the medical connector in the first position. The medical system may include a biasing member that may be sized and configured to move the medical connector to a location at which the guide no longer retains the medical connector in the first position and the medical connector resiliently moves to the second position. The medical system may include an extension that may be movable between a retracted position and an extended position to locate an opening in a patient's lumen. The medical system may include an extension that may be movable between a retracted position and an extended position, and the extension may be sized and configured to engage an inner surface of a wall of a patient's lumen when in the extended position. The at least one elongated member may be arranged in a generally ring-shaped configuration when the medical connector is in the second position. The at least one elongated member may include a plurality of elongated members; and the plurality of elongated members may be arranged in a generally sphere-shaped configuration when the medical connector is in the second position. The first and second ends may overlap when the medical connector is in the second position. The first end may contact the second end when the medical connector is in the second position.

Another aspect is a method. The method may include forming an opening in a blood vessel. The blood vessel may include a wall, and the opening may extend through an outer surface of the wall and an inner surface of the wall. The method may include closing at least a portion of the opening using a medical connector that may initially penetrate a portion of the wall disposed between the inner and outer surfaces of the wall. The method may also include, prior to the closing at least a portion of the opening using a medical connector, inserting a balloon of a medical device into the blood vessel via the opening; guiding the balloon to a position within a patient; inflating the balloon; deflating the balloon; and/or removing the balloon from the patient via the opening. The medical connector may include a first penetrating member that may initially penetrate a first portion of the wall disposed between the inner and outer surfaces of the wall; and a second penetrating member that may initially penetrate a second portion of the wall disposed between the inner and outer surfaces of the wall.

For purposes of summarizing, some aspects, advantages and features of some embodiments of the invention have been described in this summary. Not necessarily all of (or any of) these summarized aspects, advantages or features will be embodied in any particular embodiment of the invention. Some of these summarized aspects, advantages and features and other aspects, advantages and features may become more fully apparent from the following detailed description and the appended claims.

### BRIEF DESCRIPTION OF THE DRAWINGS

The appended drawings contain figures of preferred embodiments to further clarify the above and other aspects, advantages and features. It will be appreciated that these drawings depict only preferred embodiments of the invention and are not intended to limits its scope. These preferred embodiments will be described and explained with additional specificity and detail through the use of the accompanying drawings in which:

FIG. 1A is a side view of an exemplary medical device, illustrating the medical device in a fastening and/or relaxed position or state;

FIG. 1B is a side view of the medical device shown in FIG. 1A, illustrating the medical device in a deflected, deformed and/or loaded position or state;

FIG. 2A is a perspective view of another exemplary medical device, illustrating the medical device in a fastening and/or relaxed position or state;

FIG. 2B is a perspective view of the medical device shown in FIG. 2A, illustrating the medical device in a deflected, deformed and/or loaded position or state;

FIG. 3A is a side view of an exemplary medical system and an exemplary lumen, illustrating the medical device shown in FIG. 1B, a cross section of an exemplary guide, and a cross section of the lumen;

FIG. 3B is a side view of the medical system and lumen shown in FIG. 3A;

FIG. 3C is a side view of the lumen and medical device shown in FIG. 3A, illustrating an exemplary use of the medical device;

FIG. 3D is a side view of the lumen and medical device shown in FIG. 3A, illustrating another exemplary use of the medical device;

FIG. 3E is a side view of the lumen and medical device shown in FIG. 3A, illustrating yet another exemplary use of the medical device;

FIG. 4A is a side view of the medical system and lumen shown in FIG. 3A, illustrating an exemplary orientation of the medical device;

FIG. 4B is a side view of the medical system and lumen shown in FIG. 4A;

FIG. 4C is a side view of the lumen and medical device shown in FIG. 4A, illustrating an exemplary use of the medical device;

FIG. 4D is a side view of the lumen and medical device shown in FIG. 4A, illustrating another exemplary use of the medical device;

FIG. 4E is a side view of the lumen and medical device shown in FIG. 4A, illustrating yet another exemplary use of the medical device;

FIG. 5A is a side view of an exemplary embodiment of the medical system and the lumen shown in FIG. 3A, illustrating exemplary extensions in a retracted position;

FIG. 5B is a side view of the medical system and lumen shown in FIG. 4A, illustrating the extensions in an extended position; and

FIG. 6 is a front view of an extension shown in FIG. 5B.

### DETAILED DESCRIPTION OF THE PREFERRED EMBODIMENTS

The present invention is generally directed towards medical devices. The principles of the present invention, however, are not limited to medical devices. It will be understood that, in light of the present disclosure, the medical devices disclosed herein can be successfully used in connection with other types of devices.

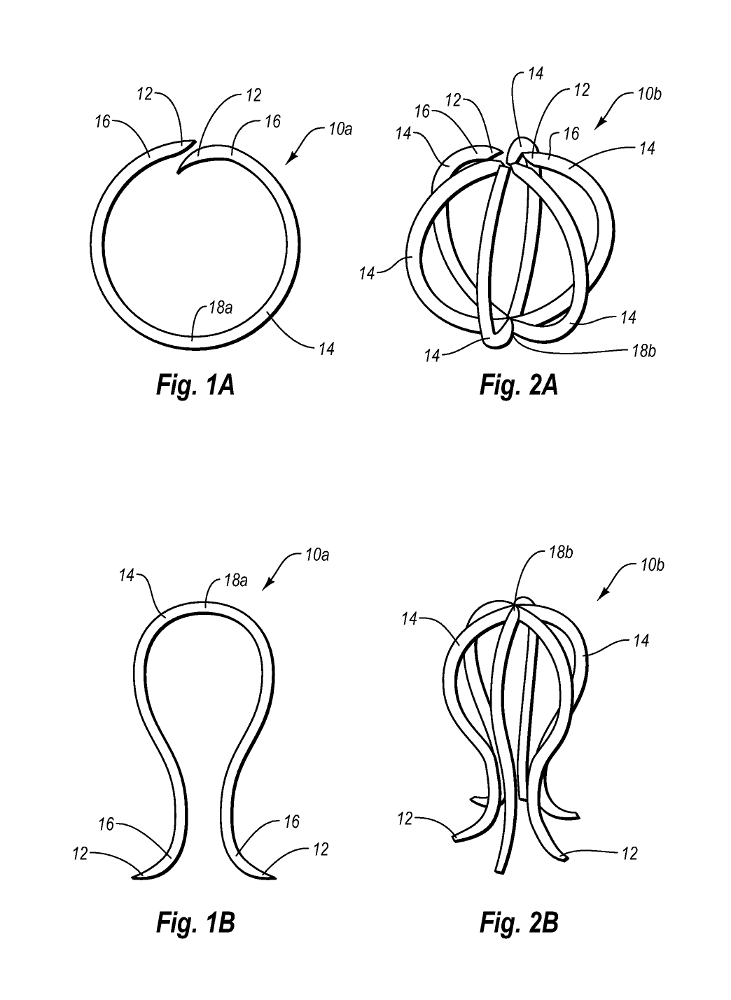

An exemplary medical device (such as medical connectors 10a, 10b shown in FIGS. 1A and 2A) may be a closure and/or any other suitable medical connector. Exemplary closures may include, for example, one or more clips, staples, and/or other suitable closures.

A medical connector 10 may be sized and configured to close an opening formed in a bodily structure of a patient. In some embodiments, the medical connector 10 may be sized and configured to close an opening formed in a lumen, such as a blood vessel. For example, in typical angioplasty procedures, an opening may be made in a patient's blood vessel (such as, as an artery in the patient's arm, groin or wrist) and another medical device (such as, a balloon catheter) may be inserted into the blood vessel via the opening. The opening may be formed by puncturing the wall of the blood vessel, by cutting the wall of the blood vessel, and/or by any other suitable method. The balloon catheter typically includes a long flexible tube and an expandable member (such as a balloon) and the balloon catheter may be guided through various blood vessels to a position in which the balloon is located within or near a narrowed portion of an artery. When in the desired position, the balloon may then be inflated to help compress plaque deposits and/or widen the artery, which may help provide improved blood flow when the balloon is deflated and the balloon catheter is removed via the opening. After the balloon catheter is removed, the opening may be closed using one or more medical connectors 10.

If desired, a small tubular device called a “stent” may be placed at or near the compressed plaque deposits and/or the widened artery. For example, in some instances, a balloon catheter may carry a stent to a desired position. When the stent is in the desired position, the balloon may then be inflated to help expand the stent. For example, at least a portion of the balloon may be positioned within the stent's passageway and the inflation may exert a force against the stent's inner wall to expand the stent. Expanding the stent may help compress the plaque deposits and/or widen the artery. Desirably, the stent may remain to help prop the artery open, which may help maintain this improved blood flow and thus increase the success rate of the angioplasty procedures. It will be appreciated, however, that the medical connector 10 need not be sized and configured to close an opening formed in a lumen or other bodily structure and that the medical connector 10 may be used for other purposes and/or in contexts other than angioplasty. In some embodiments, the medical connector 10 may be sized and configured to interconnect discrete, separate portions of one or more bodily structures. In particular, the medical connector 10 may be used for “anastomosis,” a term covering a variety of procedures in which portions of lumens (such as blood vessels, intestines, the colon, or other lumens) are joined or reconnected. Also, in some embodiments, the medical connector 10 may be sized and configured to interconnect spaced apart portions of a bodily structure to help alter the bodily structure's shape and/or configuration. In particular, the medical connector 10 may be used to create pleats or folds in bodily tissue (such as, at the gastro-oesophagal junction, which may help treat gastro-oesophagal reflux disease).

To help close an opening formed in (and/or interconnect portions of) at least one bodily structure, the medical connector 10 may include one or more penetrating members 12, which may be sized and configured to penetrate one or more portions of the at least one bodily structure. The penetrating members 12 may have a generally tapered and/or a generally pointed configuration as shown in FIG. 1A. This generally tapered and/or generally pointed configuration may better facilitate penetration into a bodily structure. It will be appreciated, however, that the penetrating members 12 do not require a generally tapered configuration or a generally pointed configuration and that the penetrating members 12 may have other shapes and/or configurations.

The medical connector 10 may include at least one elongated member 14, which may include at least one of the penetrating members 12. For example, as shown in FIG. 1A, the medical connector 10a may include a single elongated member 14, which may include first and second penetrating members 12 that may be respectively formed in or located near first and second ends 16 of the elongated member 14.

The medical connector 10 may, however, include a plurality of elongated members 14, some or all of which may include a penetrating portion 12. For example, as shown in FIG. 2A, a medical connector 10b may include six elongated members 14, which may include penetrating members 12 formed in or located near an end 16 of the elongated members. If desired, a medical connector 10 may include one, two, three, four, five, six or more such elongated members 14, and the elongated members 14 may be substantially equally spaced apart from each other. For example, each of the six elongated members 14 shown in FIG. 2A may be disposed at about a sixty degree angle relative to adjacent elongated members 14. It will be appreciated, however, that the elongated members 14 may be disposed in other relative positions, that the medical connector 10 does not require any elongated members 14, and that the medical connector 10 may include other suitable structures.

The medical connector 10 may be sized and configured to be moved to a fastening position, such as a fastening position shown in FIGS. 1A and 2A. Moving the medical connector 10 to the fastening position may help close an opening formed in at least one bodily structure and/or interconnect portions of at least one bodily structure.

The medical connector 10 may have any of a variety of different configurations when in the fastening position. For example, when the medical connector 10 is in the fastening position, the elongated members 14 may be arranged in a generally ring-shaped configuration (as shown in FIG. 1A), a generally sphere-shaped configuration (as shown in FIG. 2A), a generally spiral and/or generally helical configuration (which may better facilitate the closure and/or the interconnection provided by the medical connector 10), a generally pretzel-shaped configuration, a generally oval-shaped configuration, an American-football shaped configuration, a generally diamond shaped configuration, a generally triangle-shaped configuration, a generally pyramidal-shaped configuration, or other suitable configurations having other suitable shapes.

In addition, when the medical connector 10 is in the fastening position, the penetrating members 12 and/or the ends 16 may be disposed proximate to, may point towards, may overlap with and/or may contact each other, which may help better retain the closure and/or the interconnection provided by the medical connector 10. It will appreciated that the penetrating members 12 and/or the ends 16 need not be disposed proximate to, point towards, overlap with or contact each other when the medical connector 10 is in the fastening position. It will also be appreciated the penetrating members 12 and/or the ends 16 may be spaced apart from each other when the medical connector 10 is in the fastening position.

The medical connector 10 may be sized and configured to resiliently move to the fastening position. In particular, the medical connector 10 may be deflected, deformed and/or otherwise moved away from the fastening position. While being deflected, deformed and/or otherwise moved away from the fastening position, the medical connector 10 may be loaded with energy configured to resiliently return the medical connector 10 to the fastening position. Consequently, when the loaded energy is released, the medical connector 10 may resiliently return to the fastening position.

For example, the elongated members 14 of the medical connectors 10a, 10b may be deflected, deformed and/or otherwise moved away from a fastening position or relaxed state (such as shown in FIGS. 1A and 2A) to a second position or loaded state (such as shown in FIGS. 1B and 2B), and a force may be used to retain the elongated members 14 in the second position or loaded state. Desirably, during their movement, the elongated members 14 may be loaded with energy configured to resiliently return the elongated members 14 to the fastening position or relaxed state. Consequently, when the retaining force is removed from the elongated members 14, the loaded energy may be released, causing the elongated members 14 to resiliently return to the fastening position or relaxed state.

With reference to FIGS. 1A, 1B, 2A and 2B, as the elongated members 14 are deflected, deformed and/or otherwise moved away from the fastening position, the penetrating members 12 and/or the ends 16 may rotate or otherwise move relative to generally central portions 18a, 18b of the medical connectors 10a, 10b. This relative movement may be performed in any suitable manner. For example, the generally central portions 18a, 18b may be retained in a generally fixed location, while the penetrating members 12 and/or the ends 16 are moved. Also, the penetrating members 12 and/or the ends 16 may be retained in a generally fixed location, while the generally central portions 18a, 18b are moved.

This relative movement may evert the medical connectors 10a, 10b as shown in FIGS. 1B and 2B. As shown in FIG. 1B, when in an everted configuration, the medical 10a device may have a shape that is generally that of the capital Greek letter Omega (that is, “Ω”). When in the everted configuration, the medical connector 10a could have a generally arch-shaped configuration, a generally ring-shaped configuration or other configurations having other suitable shapes. It will be appreciated that the elongated members 14 may be deflected, deformed and/or otherwise moved in other suitable fashions and that the medical connectors 10a, 10b need not be everted when in the loaded state.

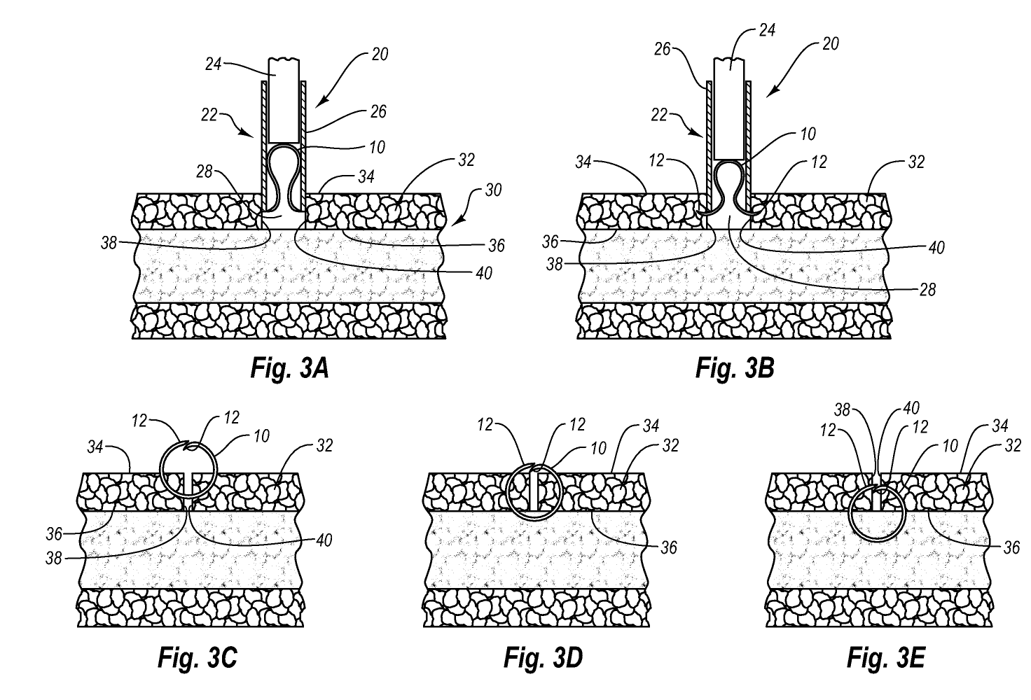

As shown in FIGS. 3A-3B, a medical system 20 may include at least one medical connector 10. The medical system 20 may also include a delivery device 22. The delivery device 22 may include a biasing member 24 and a guide 26. The biasing member 24 may include a plunger, actuator, a spring, a rod and/or other structure sized and configured to move the medical connector 10. The guide 26 may include a tube, a shaft, and/or other structure sized and configured to guide the movement of the medical connector 10.

The biasing member 24 may be movable relative to the guide 26. In particular, the guide 26 may include a hollow interior portion in which at least a portion of the biasing member 24 may be slidably or otherwise movably disposed. The hollow interior portion may extend along all or at least a portion of the length of the guide 26 and may open at one or more ends of the guide 26. If desired, a seal may be formed between biasing member 24 and the guide 26 using O-rings, other suitable structures, a compression fit and/or any other suitable means. The seal may desirably help prevent a bodily fluid, such as blood, or other substances from passing between the biasing member 24 and the guide 26.

As shown in FIG. 3A, all or at least a portion of the medical connector 10 may also be disposed within the guide 26. The guide 26 may be sized and configured to retain the medical connector 10 in a deflected, deformed and/or loaded position or state, such as shown in FIGS. 1B and 2B. In particular, the guide 26 may retain the medical connector 10 in the deflected, deformed and/or loaded position or state while the biasing member 24 pushes or otherwise moves the medical connector 10 towards, for instance, a location at which the medical connector 10 may resiliently move to the fastening and/or relaxed position or state, such as shown in FIGS. 1A and 2A. By moving to the fastening and/or relaxed position or state, the medical connector 10 may help close an opening formed in (and/or interconnect portions of) at least one bodily structure.

In further detail, as shown in FIGS. 3A-3B, an opening 28 may be formed in a lumen 30, such as a patient's blood vessel. In particular, the opening 28 may be formed through a wall 32 of the lumen 30 and may extend between an outer surface 34 and an inner surface 36 of the wall 32.

The biasing member 24 may push or otherwise move the medical connector 10 to a location at which the medical connector 10 may resiliently move to a fastening and/or relaxed position or state, such as shown in FIG. 3C. For instance, the biasing member 24 may move the medical connector 10 to a location at which at least a portion of the medical connector 10 extends beyond the end of the guide 26. In some embodiments, when at least a portion of the penetrating members 12 extend beyond the end of the guide 26, the medical connector 10 may, as shown in FIG. 3B, commence resiliently moving towards the fastening and/or relaxed position or state.

As shown in FIGS. 3B-3E, when the medical connector 10 commences resiliently moving towards the fastening and/or relaxed position or state, the penetrating members 12 may penetrate a variety of different portions of the wall 32 of the lumen 30 depending, for example, upon the particular position of the delivery device 22. In some instances, as shown in FIG. 3B, the penetrating members 12 may initially penetrate portions 38, 40 of the wall 32 located between the outer and inner surfaces 34, 36 of the wall 32 and may also, as shown in FIG. 3C, exit the wall 32 through its outer surface 34. In other instances, as shown in FIG. 3D, the penetrating members 12 may initially penetrate the inner surface 36 of the wall 32 and may also exit the wall 32 through its outer surface 34. In still other instances, as shown in FIG. 3E, the penetrating members 12 may initially penetrate the inner surface 36 of the wall 32 and may also exit the wall 32 through the portions 38, 40 located between the outer and inner surfaces 34, 36 of the wall 32.

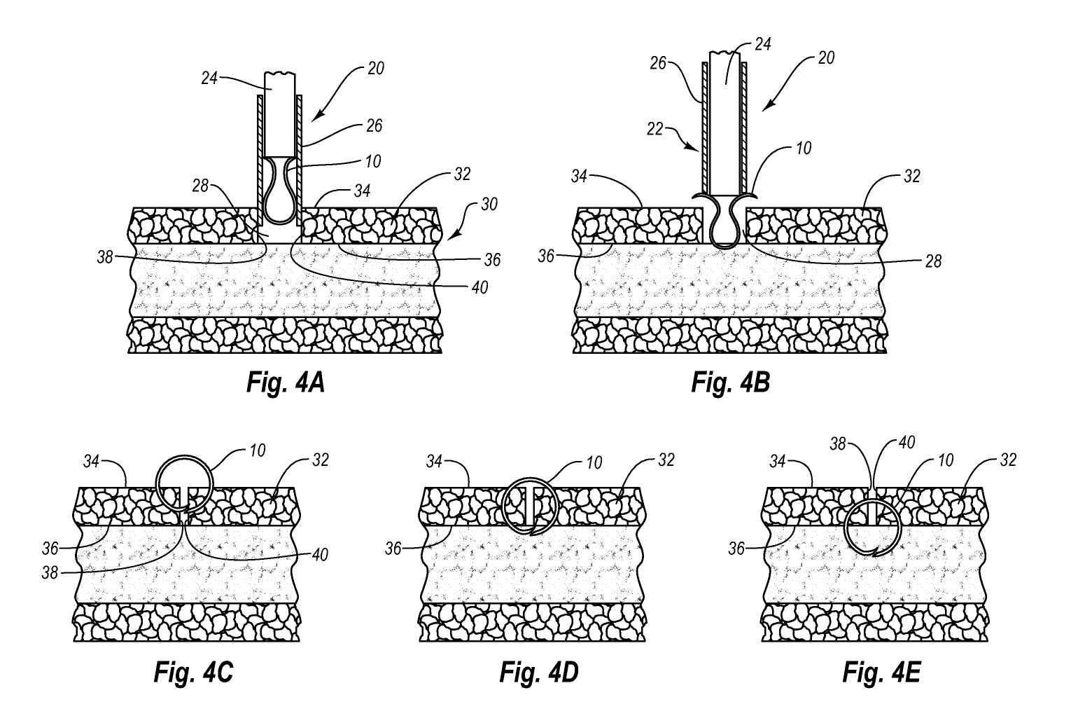

As shown in FIGS. 4B-4E, when the medical connector 10 commences resiliently moving towards the fastening and/or relaxed position or state, the penetrating members 12 may penetrate a variety of different portions of the wall 32 of the lumen 30 depending, for example, upon the particular orientation of the medical connector 10. In some instances, as shown in FIG. 4C, the penetrating members 12 may initially penetrate the outer surface 34 of the wall 32 and may also exit the wall 32 through the portions 38, 40 located between the outer and inner surfaces 34, 36 of the wall 32. In other instances, as shown in FIG. 4D, the penetrating members 12 may initially penetrate the outer surface 34 of the wall 32 and may also exit the wall 32 through its inner surface 36. In still other instances, as shown in FIG. 4E, the penetrating members 12 may initially penetrate the portions 38, 40 located between the outer and inner surfaces 34, 36 of the wall 32 and may also exit the wall 32 through its inner surface 36.

Thus, as shown above, by penetrating and/or exiting various portions of the lumen 30 and by returning to the fastening and/or relaxed position or state, the medical connector 10 may help close the opening 28 formed in the lumen 30. In particular, the medical connector 10 may engage the tissue of the lumen 30 to help pull the edges of the opening 28 together and at least partially close the opening 28, which may help reduce the amount of fluid (such as blood) and/or other substances that may leave the lumen 28. Of course, by penetrating and/or exiting various portions of one or more other bodily structures and by returning to the fastening and/or relaxed position or state, the medical connector 10 may help close an opening formed in (and/or interconnect portions of) other bodily structures having other shapes and configurations.

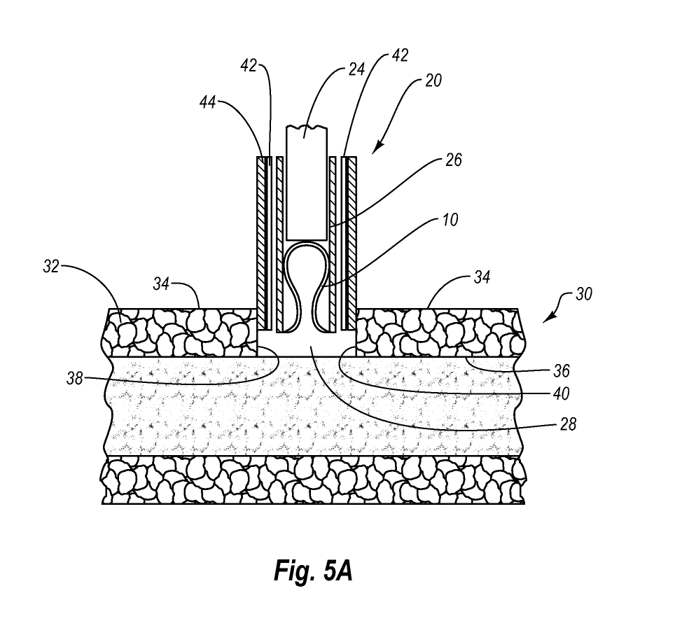

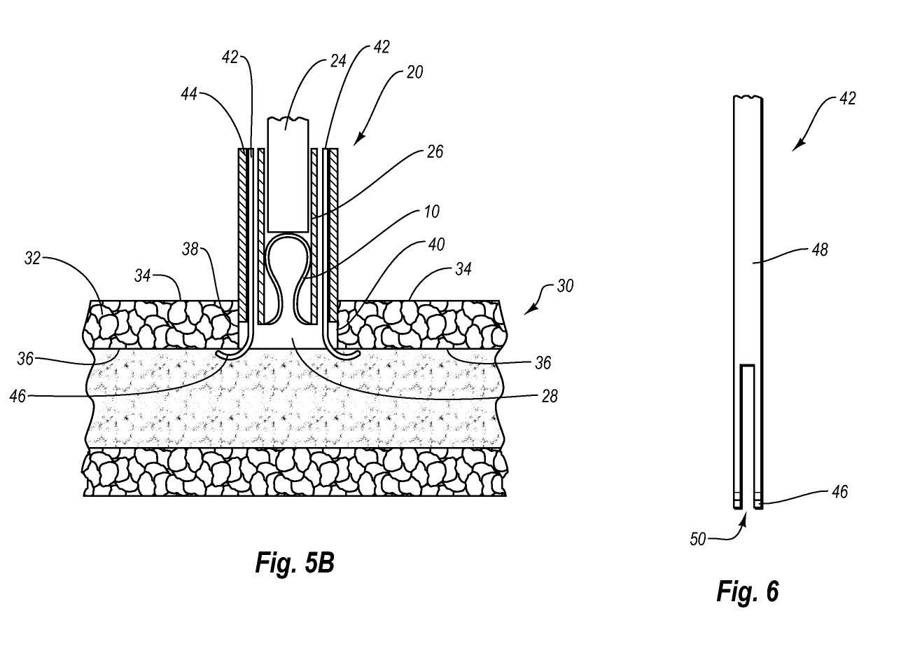

As shown in FIGS. 5A-5B, the medical system 20 may also include at least one extension 42 and a guide 44. At least a portion of the extension 42 may be disposed within the guide 44 and/or between the guides 26, 44, which may help retain the extension 42 in a desired position.

Desirably, the extension 42 may be used to help locate the opening 28 and/or to help position the guide 44 in a desired location. In particular, the extension 42 may be used to help determine whether the guide 44 is aligned with the opening 28. For example, when a person attempts to advance the extension past the end of the guide 44 and the guide 44 is not aligned with the opening 28, the extension 42 may contact and/or engage the outer surface 34 of the lumen 30, which may restrict the movement of the extension 42. Consequently, upon noticing this restricted movement, the person may realize that the guide 44 is not aligned with the opening 28 and may reposition the guide 44. In contrast, when the guide 44 is aligned with the opening 28, the extension 42 can extend into the interior of the lumen 30, after which the person may realize the guide 44 is aligned with the opening 28.

The extension 42 may include a tip 46. The extension 42 and/or the tip 46 may be constructed from a flexible and/or resilient material, for example, shape-memory materials (such as nickel-titanium alloys, known as Nitinol; other shape-memory alloys; shape memory polymers; or the like). For example, the tip 46 may be sized and configured to resiliently flex or otherwise move outwardly as it extends beyond the end of the guide 44 as shown in FIG. 5B. The outward flexing of the tips 46 may help attach the medical system 20 in a generally fixed position relative to the lumen 30.

In particular, the outward flexing of the tips 46 may allow the tips 46 to contact and/or engage the interior surface 36 of the lumen 30 to help attach the medical system 20 in a generally fixed position relative to the lumen 30. For instance, the tips 46 may contact and/or engage the interior surface 36 of the lumen 30 to help attach the medical system 20 in a generally fixed position relative to the lumen 30 that facilitates the use of the biasing member 24, the guide 26 and the medical connector 10, as described above with reference to FIGS. 1A-4E.

As shown in FIG. 6, the extension 42 may include a body 48. The body 48 may have an elongated configuration and may include one or more slots or other openings 50. Desirably, the slot 50 may be aligned with the medical connector 10, which may allow the medical connector 10 to be deployed to help close the opening 28 while the extension 42 helps keep the medical system 20 in a generally fixed position relative to the lumen 30.

After such deployment, the extension 42 may be retracted such that the tips 46 return to the position shown in FIG. 5A, and the medical system 20 may be detached from the lumen 30. Desirably, as the medical connector 10 engages the tissue of the lumen 30 to help pull the edges of the opening 28 together, and the edges of the opening 28 may cause the tip 46 to contract, which may help facilitate the retraction of the extension 42 and/or the detachment of the medical system 20. If desired, the extension 42 may be retracted prior to the deployment of the medical connector 10 and, thus, the slot 50 is not required.

The extension 42 and the tip 46 need not be constructed from a flexible or resilient material and may be constructed from other materials having other suitable characteristics. In addition, the medical system 20 and the extension 42 do not require the guide 44, and the extension 42 may have a variety of other suitable sizes, shapes and/or configurations. For example, the extension 42 may include an elongated wire, which may be disposed adjacent the guide 26.

It will be appreciated that the medical system 20 may include a variety of other suitable component and/or configurations. For example, in some embodiments, the medical system 20 may include a bleed-back tube (not shown) that may be positioned within the guide 24 to help indicate when the guide 24 is aligned with the opening 28 in the lumen 30. Also, for example, in some embodiments, the medical system 20 may include an air compressor or fluid insertion device coupled to the guide 24, which may help maintain pressure within the guide 24 to help move the medical connector 10 and/or maintain a fluid seal within the guide 24.

As shown above, the medical connector 10 may be flexible and/or resilient. Desirably, this flexibility and resiliency may allow the medical connector 10 to automatically deploy when it reaches a particular location, which may allow the medical system 20 to have a less complicated design.

In addition, this flexibility and resiliency may allow the medical connector 10 to have a smaller width when in the deflected, deformed and/or loaded position or state and a larger width when in the fastening position or state. This smaller width may advantageously allow the medical connector 10 to be disposed within a smaller guide 24. By using a smaller guide, a smaller opening 28 may be formed in the lumen 30, for example, as discussed above with respect to angioplasty procedures. This may help decrease the pain, recovery time, and/or scarring associated with the opening 28.

The medical connector 10 may be constructed from a variety of materials. In particular, the medical connector 10 may be constructed from a single material or from a plurality of materials. For example, the ends 16 of the elongated members 14 may be constructed from a first set of one or more materials, while the other portions of the elongated members 14 may be constructed from other suitable materials. If desired, the materials may be selected according to the structural performance and/or biological characteristics that are desired.

In some embodiments, the medical connector 10, the elongated members 14 and/or the ends 16 may include one or more layers of materials applied to a primary material. For example, the primary material may include a resilient and/or flexible primary material (such as, nitinol), and the one or more layers may include resilient and/or flexible materials (such as, Ti3Al2.5V, Ti6Al4V, 3-2.5Ti, 6-4Ti and platinum) that may have good crack-arresting and/or shock-absorbing characteristics and may be particularly good for adhering to a resilient and/or flexible primary material. Desirably, the primary material and the one or more layers may be configured to help the medical connector 10, the elongated members 14 and/or the ends 16 resiliently move to the fastening and/or relaxed position or state, as discussed above. In addition, the one or more layers may be useful for applying radiopaque materials to the medical connector 10, the elongated members 14 and/or the ends 16. It will be appreciated, however, that the medical connector 10, the elongated members 14 and the ends 16 do not require the layers and may have other suitable constructions using other materials having other suitable characteristics.

The primary material of the medical connector 10, the elongated members 14 and/or the ends 16 may be a shape-memory material, such as shape-memory alloys or shape-memory polymers. Desirably, the shape-memory material may have a shape memory effect in which the medical connector 10 can be made to remember an initial shape (such as, the fastening and/or relaxed position or state shown in FIGS. 1A and 2A). Once the initial shape has been remembered, the shape-memory material may be deflected, deformed and/or otherwise moved to a second shape (such as, the deflected, deformed and/or loaded position or state shown in FIGS. 1B and 2B), but may be later returned to the initial shape by heating and/or unloading the medical connector.

Typically, a shape-memory alloy can have any non-characteristic initial shape that can then be configured into a memory shape by heating the shape-memory alloy and moving the shape-memory alloy into the desired memory shape. After the shape-memory alloy is cooled, the desired memory shape can be retained. This allows for the shape-memory alloy to be bent, straightened, compacted, and placed into various contortions by the application of requisite forces; however, after the forces are released, the shape-memory alloy can be capable of returning to the memory shape. Exemplary shape-memory alloys may include, but are not limited to, copper-zinc-aluminum; copper-aluminum-nickel; nickel-titanium (“NiTi”) alloy, also known as “Nitinol”; and cobalt-chromium-nickel alloys or cobalt-chromium-nickel-molybdenum alloys known as elgiloy alloys. It will be understood that the temperatures at which the shape-memory alloy changes its crystallographic structure are characteristic of the alloy and may be tuned by varying the elemental ratios. In some embodiments, the medical connector 10, the elongated members 14 and/or the ends 16 may be constructed from a NiTi alloy that forms a Superelastic Nitinol wire. If desired, additional materials may be added to the NiTi alloy to adjust the characteristics of the Superelastic Nitinol wire. In some embodiments, the medical connector 10, the elongated members 14 and/or the ends 16 may be constructed from tubes, ribbon, stamped materials, various shapes cut from a flat sheet, and/or other structures that may be constructed from a NiTi alloy and/or any other suitable substance.

Typically, when a shape-memory polymer encounters a temperature above the lowest melting point of the individual polymers, the blend may make a transition to a rubbery state. The elastic modulus may, for instance, change more than two orders of magnitude across the transition temperature (“Ttr”). As such, a shape-memory polymer may be formed into a desired shape of a medical connector 10 by heating it above the Ttr, fixing the shape-memory polymer into the new shape, and cooling the material below Ttr. The shape-memory polymer can then be arranged into a temporary shape by force, and then resume the memory shape once the force has been removed. Exemplary shape-memory polymers include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, and vinyl acetate-polyester-based compounds.

In some embodiments, one or more layers of shape-memory polymers may be applied to the primary material. The shape-memory polymer layers may include Veriflex™, the trademark for CRG's family of shape-memory polymer resin systems. Veriflex™ currently functions on thermal activation which can be customizable from −20° F. to 520° F., allowing for customization within the normal body temperature of a patient. Desirably, a medical connector 10 including at least one Veriflex™ layer may resiliently move to the fastening and/or relaxed position or state in response to a patient's body heat and/or in response to exiting the guide 26. In one embodiment, for example, the Veriflex™ layer may help retain the medical connector 10 in the deflected, deformed and/or loaded position or state until the patient's body heat softens at least a portion of the Veriflex™ layer to permit the medical connector 10 to return the fastening and/or relaxed position or state.

In some embodiments, the medical connector 10 including at least one layer including a shape-memory material, a superelastic material and/or other suitable layers may be retained using a delivery device 22 or other restraint and then deployed to the fastening position by removal of the restraint. In some embodiments, a medical connector 10 may be constructed from a thermally-sensitive material (such as, a shape-memory polymer) and may be deployed by exposure of the medical connector 10 to a sufficient temperature to facilitate deployment.

In some embodiments, the medical connector 10 may include a variety of known suitable deformable materials, including stainless steel, silver, platinum, tantalum, palladium, cobalt-chromium alloys or other known biocompatible materials, which may be fashioned from a wire, sheet, or other material configuration.

In some embodiments, the medical connector 10 may include a suitable biocompatible material, such as a biocompatible polymer. Exemplary biocompatible materials can include a suitable hydrogel, hydrophilic polymer, biodegradable polymers, bioabsorbable polymers. Exemplary polymers can include poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, combinations thereof, or the like.

In some embodiments, the medical connector 10 may include a ceramic material. For instance, the ceramic can be a biocompatible ceramic which optionally can be porous. Exemplary ceramic materials may includes hydroxylapatite, mullite, crystalline oxides, non-crystalline oxides, carbides, nitrides, silicides, borides, phosphides, sulfides, tellurides, selenides, aluminum oxide, silicon oxide, titanium oxide, zirconium oxide, alumina-zirconia, silicon carbide, titanium carbide, titanium boride, aluminum nitride, silicon nitride, ferrites, iron sulfide, and the like. In some embodiments, the ceramic can be provided as sinterable particles that are sintered into the shape of the medical connector 10 or portion or layer thereof

In some embodiments, the medical connector 10 may include a radiopaque material to increase visibility during placement. For instance, the radiopaque material can be a layer or coating applied to at least a portion of the medical connector 10. The radiopaque materials can be platinum, tungsten, silver, stainless steel, gold, tantalum, bismuth, barium sulfate, or other radiopaque materials.

In some embodiments, the medical connector 10 may include biodegradable or bioabsorbable materials.

In some embodiments, the medical connector 10 may include absorbent biomaterials. Exemplary biomaterials may include, but are not limited to, lyophilized or air-dried submucosal tissue or other extracellular matrix-derived tissue from warm-blooded vertebrate. Such materials have a variety of characteristics, including one or more of: biological remodeling, resistance to infection, and high similarity to autogenous material. Examples of such submucosal or other extracellular matrix-derived tissue is described in U.S. Pat. Nos. 4,902,508, 5,281,422, 5,573,784, 5,573,821, 6,206,931, and 6,790,220, the disclosures of which are incorporated by reference herein.

In some embodiments, the medical connector 10 may include a coating of biomaterial around a core forming the medical connector 10. As such a coating can have high similarity to autogenous material of the patient, there can be a reduced risk that the patient will reject the closure device or receive an infection. Moreover, biological remodeling characteristics of matrix-derived biomaterials can further foster regeneration of tissue around the medical connector 10 to close the opening 28 and thereby prevent excess bodily fluid loss. In some embodiments, the core may be constructed from wires, tubes, ribbon, stamped materials, various shapes cur from a flat sheet, and/or other structures that may be constructed from a NiTi alloy and/or any other suitable substance.

Although this invention has been described in terms of certain preferred embodiments, other embodiments apparent to those of ordinary skill in the art are also within the scope of this invention. Accordingly, the scope of the invention is intended to be defined only by the claims which follow.

What is claimed is:

1. A medical system comprising:
a medical connector including at least one elongated member, the medical connector being sized and configured to resiliently move from an everted position to a fastening position.

2. The medical system as in claim 1, wherein the at least one elongated member is arranged in a generally ring-shaped configuration when the medical connector is in the fastening position.

3. The medical system as in claim 1, wherein the at least one elongated member includes a plurality of elongated members; and wherein the plurality of elongated members are arranged in a generally sphere-shaped configuration when the medical connector is in the fastening position.

4. The medical system as in claim 1, further comprising a guide sized and configured to retain the medical connector in the everted position.

5. The medical system as in claim 4, further comprising a biasing member sized and configured to move the medical connector to a location at which the guide no longer retains the medical connector in the everted position and the medical connector resiliently moves to the fastening position.

6. The medical system as in claim 1, further comprising an extension movable between a retracted position and an extended position to locate an opening in a patient's lumen.

7. The medical system as in claim 1, further comprising an extension movable between a retracted position and an extended position, the extension being sized and configured to engage an inner surface of a wall of a patient's lumen when in the extended position.

8. The medical system as in claim 1, wherein the at least one elongated member includes a first penetrating member and a second penetrating member; and wherein the first and second penetrating members face generally towards each other when the medical connector is in the fastening position and face generally away from each other when the medical connector is in the everted position.

9. The medical system as in claim 1, wherein the at least one elongated member includes a first end and a second end; and wherein the first and second ends face generally towards each other when the medical connector is in the fastening position and face generally away from each other when the medical connector is in the everted position.

10. The medical system as in claim 1, wherein the at least one elongated member includes a first penetrating member and a second penetrating member; and wherein the first and second penetrating members overlap when the medical connector is in the fastening position.

11. The medical system as in claim 1, wherein the at least one elongated member includes a first penetrating member and a second penetrating member; and wherein the first penetrating member contacts the second penetrating member when the medical connector is in the fastening position.

12. The medical system as in claim 1, wherein the at least one elongated member includes a first end and a second end; and wherein the first and second ends overlap when the medical connector is in the fastening position.

13. The medical system as in claim 1, wherein the at least one elongated member includes a first end and a second end; and wherein the first end contacts the second end when the medical connector is in the fastening position.

14. A medical system comprising:
a medical connector including at least one elongated member, the at least one elongated member including a first penetrating member and a second penetrating member, the medical connector being sized and configured to resiliently move from a first position to a second position, the first and second penetrating members facing generally towards each other when the medical connector is in the second position, the first and second penetrating members facing generally away from each other when the medical connector is in the first position.

15. The medical system as in claim 14, further comprising a guide sized and configured to retain the medical connector in the first position.

16. The medical system as in claim 15, further comprising a biasing member sized and configured to move the medical connector to a location at which the guide no longer retains the medical connector in the first position and the medical connector resiliently moves to the second position.

17. The medical system as in claim 14, further comprising an extension movable between a retracted position and an extended position to locate an opening in a patient's lumen.

18. The medical system as in claim 14, further comprising an extension movable between a retracted position and an extended position, the extension being sized and configured to engage an inner surface of a wall of a patient's lumen when in the extended position.

19. The medical system as in claim 14, wherein the at least one elongated member is arranged in a generally ring-shaped configuration when the medical connector is in the second position.

20. The medical system as in claim 14, wherein the at least one elongated member includes a plurality of elongated members; and wherein the plurality of elongated members are arranged in a generally sphere-shaped configuration when the medical connector is in the second position.

21. The medical system as in claim 14, wherein the first and second penetrating members overlap when the medical connector is in the second position.

22. The medical system as in claim 14, wherein the first penetrating member contacts the second penetrating member when the medical connector is in the second position.

23. A medical system comprising:
a medical connector including at least one elongated member, the at least one elongated member including a first end and a second end, the medical connector being sized and configured to resiliently move from a first position to a second position, the first and second ends facing generally towards each other when the medical connector is in the second position, the first and second ends facing generally away from each other when the medical connector is in the first position.

24. The medical system as in claim 22, further comprising a guide sized and configured to retain the medical connector in the first position.

25. The medical system as in claim 24, further comprising a biasing member sized and configured to move the medical connector to a location at which the guide no longer retains the medical connector in the first position and the medical connector resiliently moves to the second position.

26. The medical system as in claim 22, further comprising an extension movable between a retracted position and an extended position to locate an opening in a patient's lumen.

27. The medical system as in claim 22, further comprising an extension movable between a retracted position and an extended position, the extension being sized and configured to engage an inner surface of a wall of a patient's lumen when in the extended position.

28. The medical system as in claim 22, wherein the at least one elongated member is arranged in a generally ring-shaped configuration when the medical connector is in the second position.

29. The medical system as in claim 22, wherein the at least one elongated member includes a plurality of elongated members; and wherein the plurality of elongated members are arranged in a generally sphere-shaped configuration when the medical connector is in the second position.

30. The medical system as in claim 22, wherein the first and second ends overlap when the medical connector is in the second position.

31. The medical system as in claim 22, wherein the first end contacts the second end when the medical connector is in the second position.

32. A method comprising:
forming an opening in a blood vessel, the blood vessel including a wall, the opening extending through an outer surface of the wall and an inner surface of the wall; and
closing at least a portion of the opening using a medical connector that initially penetrates a portion of the wall disposed between the inner and outer surfaces of the wall.

33. The method as in claim 32, further comprising, prior to the closing at least a portion of the opening using a medical connector:
inserting a balloon of a medical device into the blood vessel via the opening;
guiding the balloon to a position within a patient;
inflating the balloon;
deflating the balloon; and
removing the balloon from the patient via the opening.

34. The method as in claim 32, further wherein the medical connector includes:
a first penetrating member that initially penetrates a first portion of the wall disposed between the inner and outer surfaces of the wall; and
a second penetrating member that initially penetrates a second portion of the wall disposed between the inner and outer surfaces of the wall.
